(19) United States
(12) Patent Application Publication
(10) Pub. No.: US 2006/0241600 A1
(43) Pub. Date: Oct. 26, 2006

(54) Percutaneous pedicle screw assembly

(76) Inventors: Michael D. Ensign, Salt Lake City, UT (US); Thomas M. Sweeney II, Sarasota, FL (US)

Correspondence Address: STEVEN L. NICHOLS;RADER, FISHMAN & GRAVER PLLC, 10653 S. RIVER FRONT PARKWAY, SUITE 150, SOUTH JORDAN, UT, 84095, US

(21) Appl. No.: 11/388,567

(22) Filed: Mar. 23, 2006

Related U.S. Application Data
(60) Provisional application No. 60/665,032, filed on Mar. 23, 2005.
(60) Provisional application No. 60/741,653, filed on Dec. 2, 2005.

(51) Int. Cl. A61F 2/30

(52) U.S. Cl. 606/61

(57) ABSTRACT

An exemplary connection member for percutaneously coupling to one or more orthopedic fasteners includes a tulip assembly, and, a rod, wherein the rod is permanently coupled to the tulip assembly. According to another embodiment, a connection member for percutaneously coupling to one or more orthopedic fasteners includes a fastener head securing member including a fastener head securing orifice having an axis defined by a wall member terminating in a seating member, an adjustable compression member coupled to a surface of the wall member, a rod coupled to the wall member, and a fastener head receiving orifice formed in the wall member, wherein the fastener head receiving orifice is formed transverse to and intersects the screw head securing orifice axis.

### RELATED APPLICATIONS

This application claims the benefit under 35 U.S.C. § 119(e) of U.S. Provisional Patent Application No. 60/665,032 filed Mar. 23, 2005, titled “Percutaneous Pedicle Screw System,” and U.S. Provisional Patent Application No. 60/741,653 filed Dec. 2, 2005, titled “Open End Percutaneous Screw Assembly.” The provisional applications are incorporated herein by reference in their entireties.

### TECHNICAL FIELD

The present exemplary system and method relates to medical devices. More particularly, the present exemplary system and method relates to percutaneous orthopedic rod placement devices.

### BACKGROUND

The use of bone stabilization/fixation devices to align or position bones is well established. Furthermore, the use of spinal bone stabilization/fixation devices to align or position specific vertebrae or a region of the spine is well established. Typically such devices for the spine utilize a spinal fixation element, comprised of a relatively rigid member such as a plate, a board, or a rod that is used as a coupler between adjacent vertebrae. Such a spinal fixation element can effect a rigid positioning of adjacent vertebrae when attached to the pedicle portion of the vertebrae using pedicle bone anchorage screws. Once the coupled vertebrae are spatially fixed in position, procedures can be performed, healing can proceed, or spinal fusion may take place.

Spinal fixation elements may be introduced to stabilize the various vertebrae of the spine. Some devices for this purpose are designed to be attached directly to the spine, but the generally invasive nature of standard paraspinal approach used to implant these devices may pose drawbacks. For example, muscle disruption and blood loss may result from standard paraspinal implantation approaches.

Conventional pedicle screw systems and even more recently designed pedicle screw systems also have several drawbacks. Some of these pedicle screw systems are rather large and bulky, which may result in more tissue damage in and around the surgical site when the pedicle screw system is installed during surgery. The prior art pedicle screw systems have a rod-receiving device that is pre-operatively coupled or attached to the pedicle screw. In addition, some of the prior art pedicle screw systems include numerous components that must all be carefully assembled together. Further, traditional pedicle screw systems are pre-operatively assembled, which makes these systems more difficult to install and maneuver in a spinal operation where MIS techniques are used.

### SUMMARY

In one of many possible embodiments, the present exemplary system provides a connection member for coupling to one or more pedicle screws including a tulip member having a screw head securing orifice defined by a wall member terminating in a seating member, a set screw member coupled to a surface of the wall member, a rod coupled to the wall member, and a pedicle screw head receiving orifice formed in the wall member, wherein the pedicle screw head receiving orifice is formed transverse to and intersects the screw head securing orifice.

Another exemplary embodiment provides a pedicle screw system including a pedicle screw, a tulip assembly, and a connector rod. According to this exemplary embodiment, the tulip assembly includes an outer tulip, a split ring and a saddle disposed in the outer tulip, and a set screw. Further, the connector rod includes a rod and a removable ball end disposed on one end of the connector rod. According to this exemplary embodiment, the tulip assembly and the rod may be percutaneously inserted into a patient. Further, the rod may be subcutaneously rotated to align with a plurality of pedicle screw assemblies.

Another embodiment of the present exemplary system and method provides a method for coupling a connection member to a pedicle screw including inserting a head of a pedicle screw through a first orifice in the connection member along a first line of motion, orienting the connection member with respect to the pedicle screw such that the screw shaft is oriented perpendicular to the first line of motion, seating the screw head in the connection member, and securing the position of the pedicle screw in the connection member.

### BRIEF DESCRIPTION OF THE DRAWINGS

The accompanying drawings illustrate various embodiments of the present system and method and are a part of the specification. The illustrated embodiments are merely examples of the present system and method and do not limit the scope thereof.

FIG. 1 is an exploded perspective view of a percutaneous connection member, according to one exemplary embodiment.

FIG. 2 is a perspective view of a percutaneous connection member, according to one exemplary embodiment.

FIGS. 3A, 3B, 3C, and 3D are respectively front, top, side cross-sectional, and bottom views of the percutaneous connection member of FIG. 2, according to a number of exemplary embodiments.

FIG. 4 is a flow chart illustrating a percutaneous placement method, according to one exemplary embodiment.

FIGS. 5A through 5L illustrate a tulip first percutaneous placement method, according to one exemplary embodiment

FIG. 6 illustrates the steps of a tulip first placement method, according to one exemplary embodiment.

FIGS. 7A through 7D illustrate a tulip first placement method, according to another exemplary embodiment.

FIGS. 8A through 10C illustrate the mechanics of engaging the exemplary percutaneous connection member illustrated in FIG. 2 on the head of a pedicle screw, according to one exemplary embodiment.

FIG. 11 illustrates the steps of a rod first placement method, according to one exemplary embodiment.

FIGS. 12A through 12C illustrate a rod first placement method, according to one exemplary embodiment.

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings. Throughout the drawings, identical reference numbers designate similar but not necessarily identical elements.

### DETAILED DESCRIPTION

The present specification provides a number of exemplary connection members and methods that can be used for any number of orthopedic rod placement systems. According to the present exemplary system and method, pecutaneous screw placement is facilitated. Specifically, the present exemplary systems and methods provide for the percutaneous placement of pedicle screws, followed by easy placement of the rod and one or more tulips simultaneously via a percutaneous tube. As will be described in further detail below, the present exemplary connection member may be percutaneously inserted either rod first, or tulip first. Furthermore, due to the fixed connection between the rod and the tulip of one exemplary system configuration, the profile and volume of the present exemplary system are reduced, when compared to traditional systems.

By way of example, pedicle screw systems may be fixed in the spine in a posterior lumbar fusion process via minimally invasive surgery (MIS) techniques. The systems are inserted into the pedicles of the spine and then interconnected with rods to manipulate (e.g., correct the curvature, compress or expand, and/or structurally reinforce) at least portions of the spine. Using the MIS approach to spinal fixation and/or correction surgery has been shown to decrease a patient's recovery time and reduce the risks of follow-up surgeries.

Traditional percutaneous fixation techniques are really only percutaneous in name. That is, they still require significant paraspinous tissue damage in order to fixedly couple a connector rod between two or more tulips. This is due in part to the implants that are available to the surgeon. The present exemplary system and method allows a surgeon to place spinal screws and rods via a true percutaneous approach by providing for pivoting of the rod beneath the skin in a fascial plane, lateral to the multifidous.

The ability to efficiently perform spinal fixation and/or correction surgeries using MIS techniques is enhanced by the use of pedicle screw systems provided in accordance with the present exemplary systems and methods, which systems and methods provide a number of advantages over conventional systems. For example, a pedicle screw system in accordance with one embodiment of the present exemplary system and method provides the advantage that the pedicle screw may be inserted into the bone without being pre-operatively coupled with the rod-coupling assembly (hereinafter referred to as a tulip assembly). This is advantageous because the surgeon often needs to do other inter-body work after inserting the pedicle screw, but before attaching the larger and bulkier tulip assembly. Such an advantageous pedicle screw system may be even more crucial when using MIS techniques because the inter-body spatial boundaries in which the surgeon must work may be quite limited.

The term “distraction,” when used herein and when used in a medical sense, generally relates to joint surfaces and suggests that the joint surfaces move perpendicular to one another. However when “traction” and/or “distraction” is performed, for example on spinal sections, the spinal sections may move relative to one another through a combination of distraction and gliding, and/or other degrees of freedom.

In the following description, certain specific details are set forth in order to provide a thorough understanding of various embodiments of the present percutaneous pedicle screw system. However, one skilled in the relevant art will recognize that the present exemplary system and method may be practiced without one or more of these specific details, or with other methods, components, materials, etc. In other instances, well-known structures associated with pedicle screws have not been shown or described in detail to avoid unnecessarily obscuring descriptions of the embodiments of the systems and methods.

Unless the context requires otherwise, throughout the specification and claims which follow, the word “comprise” and variations thereof, such as, “comprises” and “comprising” are to be construed in an open, inclusive sense, that is as “including, but not limited to.”

Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment. Furthermore, the particular features, structures, or characteristics may be combined in any suitable manner in one or more embodiments.

Exemplary Structure

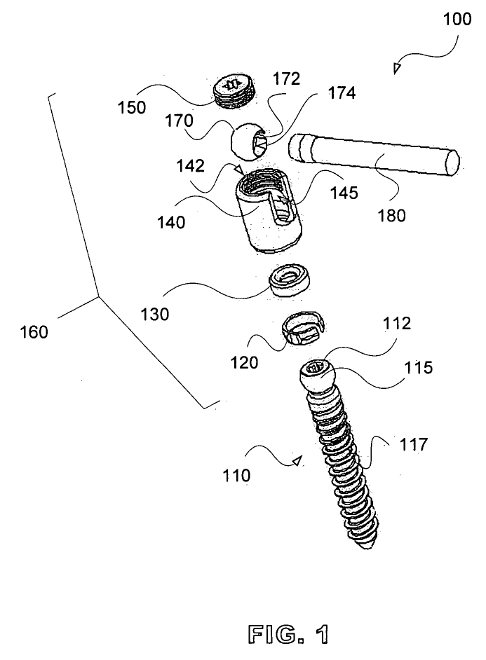

FIG. 1 is an exploded perspective view illustrating the components of a percutaneous pedicle screw system (100), according to one exemplary embodiment. As illustrated in FIG. 1, the exemplary percutaneous pedicle screw system (100) includes a pedicle screw (110) having a head portion (115). According to the exemplary embodiment illustrated in FIG. 1, the pedicle screw (110) includes an elongated, threaded portion (117) and a head portion (115). Although pedicle screws (110) are generally known in the art, the head portions (115) may be of varying configurations depending on what type of tulip assembly is to be coupled to the pedicle screw (110). The head portion (115) of the present exemplary pedicle screw (110) includes a driving feature (112) and a maximum diameter portion. The driving feature (112) of the present exemplary pedicle screw (110) permits the screw to be inserted into a pedicle bone and/or other bone. According to one exemplary embodiment, the pedicle bone is a part of a vertebra that connects the lamina with a vertebral body. Additionally, according to the present exemplary embodiment, the driving feature (112) can be used to adjust the pedicle screw (110) prior to or after the tulip assembly is coupled to the pedicle screw (110). In the illustrated embodiment, the head portion (115) of the pedicle screw (110) is coupled to the threaded portion (117) and includes a generally spherical surface with a truncated or flat top surface.

In one exemplary embodiment, the pedicle screw (110) is cannulated, which means a channel (not shown) extends axially through the pedicle screw (12)) extends through the entire length of the pedicle screw (110). The channel (not shown) allows the pedicle screw (110) to be maneuvered over and receive a Kirschner wire, commonly referred to as a K-wire. The K-wire is typically pre-positioned using imaging techniques, for example, fluoroscopy imaging, and then used to provide precise placement of the pedicle screw (110). While the pedicle screw (110) illustrated in FIG. 1 includes a number of components, numerous variations may be made including, but in no way limited to, varying the type of driving feature (112), varying the head shape, varying materials, varying dimensions, and the like.

In addition to the exemplary pedicle screw (110), the exemplary percutaneous pedicle screw system (100) includes a tulip assembly (160) that may be coupled to the head portion (115) of the pedicle screw (110) after the pedicle screw has been percutaneously inserted into a desired pedicle, while allowing for an orientation of a connector rod (180) beneath a patient's skin. As illustrated in FIG. 1, the tulip assembly (160) includes a main tulip housing (140) containing a split ring (120) and a saddle (130) element disposed in a lower portion thereof. Additionally, a ball end (170) and a set screw (150) may be selectively assembled in the upper portion of the tulip housing (140). Moreover, as shown, material is removed from the sidewall of the tulip housing (140) to form a rod cut-out (145). Further, a connector rod (180) is selectively inserted into the tulip assembly (160). Further details of the exemplary tulip assembly (160) will be provided below.

As shown, the tulip housing (140) includes an inner bore (142) that extends concentrically along the axis of the cylindrically shaped tulip housing. As shown, a split ring (120) and a saddle (130) are disposed in the lower portion of the tulip housing (140). According to one exemplary embodiment, the positioning of the split ring (120) and the saddle (130) in the lower portion of the tulip housing (140), in connection with the profile of the inner bore (142) allows the tulip assembly (160) to be snapped onto the head portion (115) of a pedicle screw (110) after the pedicle screw has been secured to a bony feature, as is described in detail in U.S. patent application Ser. No. 11/327,132 filed on Jan. 6, 2006, titled “Bone Fixation System and Method for Using the Same,” which reference is incorporated herein by reference, in its entirety. According to one exemplary embodiment, the tulip housing (140) includes a ring expansion channel and a tapered retention bore formed in the inner bore (142) configured to interact with the split ring fastener (120) during reception and fixation of the head portion (115) of the pedicle screw (110). According to one exemplary embodiment, the ring expansion channel (not shown) has a maximum diameter sufficiently large to receive the split ring fastener (120) and accommodate expansion of the split ring fastener as it receives the head portion (115) of the pedicle screw (110). Moreover, the saddle (130) may interact with the top portion of the head (115) to positional secure the head portion of the pedicle screw (110) there between. Additionally, a tapered retention bore may be formed in the expansion channel. The as detailed in the incorporated application, the tapered retention bore is configured to interact with a seating taper of the split ring fastener (120). According to one exemplary embodiment, the tulip assembly (160) may be positionally fixed relative to the pedicle screw (110), at least partially, by forcing the split ring fastener (120) along the tapered retention bore (not shown). According to one exemplary embodiment, interaction between the tapered retention bore and the seating taper constricts the split ring fastener (120) about the head portion (115) of the pedicle screw (110), positionally fixing the tulip assembly (160) relative to the pedicle screw.

Turning to the structure of the tulip housing (140), the tulip housing defines an inner bore (142) and a rod cut out (145) formed in the side of the tulip housing. According to one exemplary embodiment, the inner bore (142) may have a number of features and operational surface variations formed therein. For example, as mentioned above, the lower portion of the inner bore (142) may include a number of varying diameters to house the split ring (120) and saddle (130) members and allow their operational translations and expansions. Additionally, according to the exemplary embodiment illustrated in FIG. 1, the inner bore (142) of the tulip housing (140) may include a threaded portion configured to matingly receive the set screw (150). Additionally, the inner bore (142) may include a chamber configured to accept the ball end (170). Additionally, as shown, a rod cutout (145) may be formed in a sidewall of the tulip housing (140). According to one exemplary embodiment, the rod cutout (145) is sized to allow for rotation of a connector rod (180) from a position concentric with the axis of the inner bore (140) to a position perpendicular thereto. Consequently, according to one exemplary embodiment, the rod cutout (145) is approximately as wide as the largest diameter of the connector rod (180), according to one exemplary embodiment.

As mentioned, a ball end (170) may be disposed within the inner bore (142) of the tulip housing (140). According to one exemplary embodiment, the ball end (170) includes a center bore (172) and an expansion split (174) formed in the side wall thereof. According to one exemplary embodiment, the center bore (172) has a diameter substantially equal to or slightly smaller than the outer diameter of the connector rod (180). According to this exemplary embodiment, when the rod is inserted into the center bore (172) of the ball end (170), the ball end may expand, due to the expansion split (174), and compressibly couple the connector rod (180). Additionally, corresponding features on the end of the connector rod (180) and the split ball end (170), such as apposing tapers, single or multiple radial grooves, threading or any other features may also be used to maintain the connector rod and the ball end engaged. According to one exemplary embodiment, the ball end (170) is configured to be coupled to the connector rod (180) as described above and facilitate rotation of the connector rod within the inner bore (142) of the tulip housing (140).

Further, the set screw (150) is configured to matingly engage the internal threads formed on the inner bore (142) to compress the ball end (170) and the connector rod (180) when they are in a desired position. This will positionally secure the connector rod relative to the tulip assembly (160). Additionally, as will be described in further detail below, advancement of the set screw (150) in the inner bore (142) will impart a compressive force through the ball end (170) to the saddle (130). Consequently, the saddle (130) will further seat the split ring (120) within the tapered retention bore, either by directly forcing the split ring into the tapered bore via contact or indirectly forcing the split ring into the tapered bore by forcing the head of the pedicle screw downward, further coupling the tulip assembly (160) on the head (115) of the pedicle screw (110).

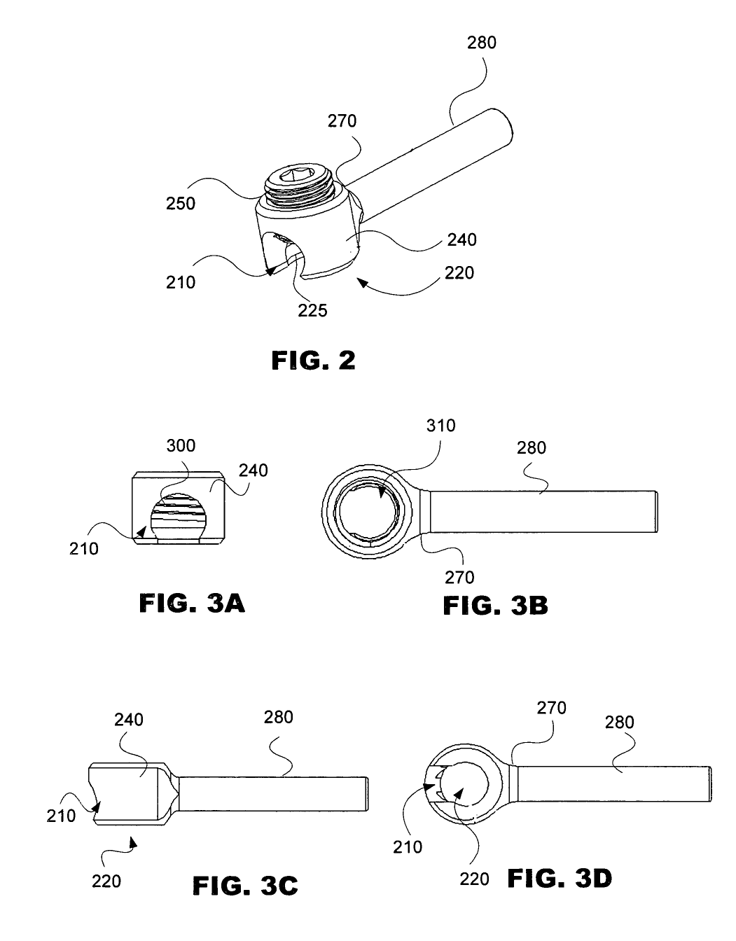

FIG. 2 illustrates an alternative percutaneous pedicle screw structure (200) according to one alternative embodiment. As illustrated in FIG. 2, the alternative percutaneous pedicle screw structure (200) includes a tulip housing (240) permanently coupled to the rod (280) by a rod coupling feature (270). Additionally, the tulip housing includes a number of features that facilitate reception, rotation, and coupling of a head portion (115) of a pedicle screw (110), according to one exemplary embodiment. As illustrated in FIG. 2, the exemplary tulip housing includes a head reception orifice (210) formed in the side wall of the tulip housing (240). Further, an exit bore (220) is formed concentric with the axis of the cylindrically shaped tulip housing (240). As shown, a seating taper (225) is formed on the inner surface of the exit bore (220). Further, a set screw (250) is axially coupled to the tulip housing (240). Further details of the alternative percutaneous pedicle screw structure will be provided below with reference to FIGS. 2 through 3D.

As mentioned, the alternative percutaneous pedicle screw structure (200) includes the rod (280) securely coupled to the side wall of the tulip housing (240) by a rod coupling feature (270). According to one exemplary embodiment, the alternative percutaneous pedicle screw system (200), the rod (280) may be securely coupled to the tulip housing (240) because the side head reception orifice (210) is leveraged to eliminate a need for rotation of the rod (280) independent of the tulip housing (240), as will be described in detail below. According to one exemplary embodiment, the rod (280) may be coupled to the side wall of the tulip housing (240) using any number of joining methods known in the art including, but in no way limited to, welding, brazing, or the use of adhesives. Alternatively, the rod coupling feature (270) may include any number of mechanical joining features including, but in no way limited to, a threaded engagement feature or an interference press fit feature.

As best seen in FIG. 3A, the head reception orifice (210) is formed in the side wall of the tulip housing (240), according to one exemplary embodiment. The head reception orifice (210) corresponds in size and shape to the head portion (115) of the pedicle screw (110). Accordingly, the head portion (115) of the pedicle screw (110) may be received by the head reception orifice (210) along any number of entry angles. Specifically, the exemplary tulip housing (240) may approach the head portion (115) of the pedicle screw (110) from a direction parallel to the axis of the pedicle screw, perpendicular to the axis of the pedicle screw, or any other direction relative to the axis of the pedicle screw, as may be dictated by the circumstances of the surgery or the preferences of a surgeon. Consequently, the head reception orifice (210) is sized to receive any profile of the head portion (115) of the pedicle screw (110).

Continuing with FIGS. 2 through 3D, the tulip housing (240) includes a thru-bore (310) passing through the entire tulip housing concentric with the axis of the housing, as seen in FIG. 3B. The upper portion of the thru-bore may include any number of internal threads or other mating features to securely mate with the set screw (250). The thru-bore (310) terminates at the bottom orifice (220). According to one exemplary embodiment, the bottom orifice (220) has a largest diameter that is smaller than the largest diameter of the head portion (115) of the pedicle screw (110), but greater than the outer diameter of the thread portion (117). Consequently, once the head portion (115) has entered the thru-bore (310) via the head reception orifice (210), it will not be released through the bottom orifice (220). However, the bottom orifice (220) may include a seating taper (225) to seat the lower surface of the head portion (115) of the pedicle screw (110).

According to one exemplary embodiment, when the head portion (115) of a pedicle screw (110) is received, via the head reception orifice (210), and the percutaneous pedicle screw system (200) has been properly positioned, the set screw (250) may be advanced along the thru-bore to positionally secure the exemplary percutaneous pedicle screw system. Specifically, when advanced along the thru-bore (310), the set screw (250) will force the head portion (115) of the pedicle screw (110) to seat in the seating taper (225) of the bottom orifice (220). According to this exemplary embodiment, forcing the head portion (115) of the pedicle screw (110) into the seating taper (225) will positionally secure the tulip housing (240) and the rod (280) relative to the pedicle screw. Additionally, by advancing the set screw (250) sufficiently along the thru-bore (310), the head reception orifice (210) will be reduced to prevent the head portion (115) of the pedicle screw (110) from exiting the tulip housing (240). According to one exemplary embodiment, the set screw (250) may include a concave surface on the underside thereof configured to matingly receive the head portion (115) of the pedicle screw (110) when engaged.

Both of the illustrated percutaneous pedicle screw systems (100, 200) are configured to provide elegant solutions to maintaining polyaxial movement in the orthopedic rod placement system. Additionally, both exemplary systems may be used to perform a truly percutaneous rod placement according to MIS insertion methods, as will be described in detail below.

Exemplary Method and Operation

While the exemplary percutaneous pedicle screw systems (100, 200) described above may be used in traditional orthopedic applications, the current exemplary methods and operations will be described, for ease of explanation only, in the context of percutaneous rod placement methods using MIS techniques. Nevertheless, it will be understood that various modifications may be made without departing from the spirit and scope of the present exemplary systems and methods.

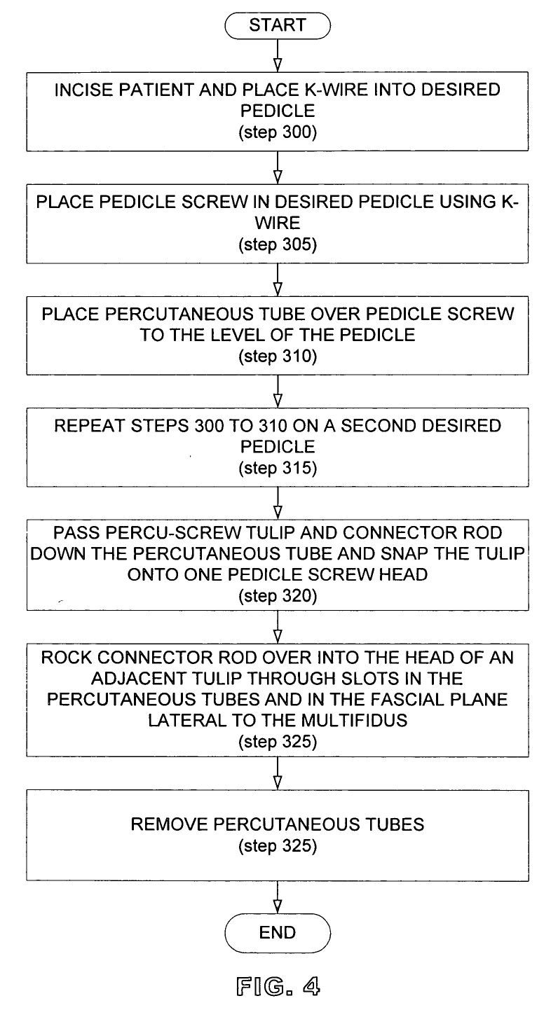

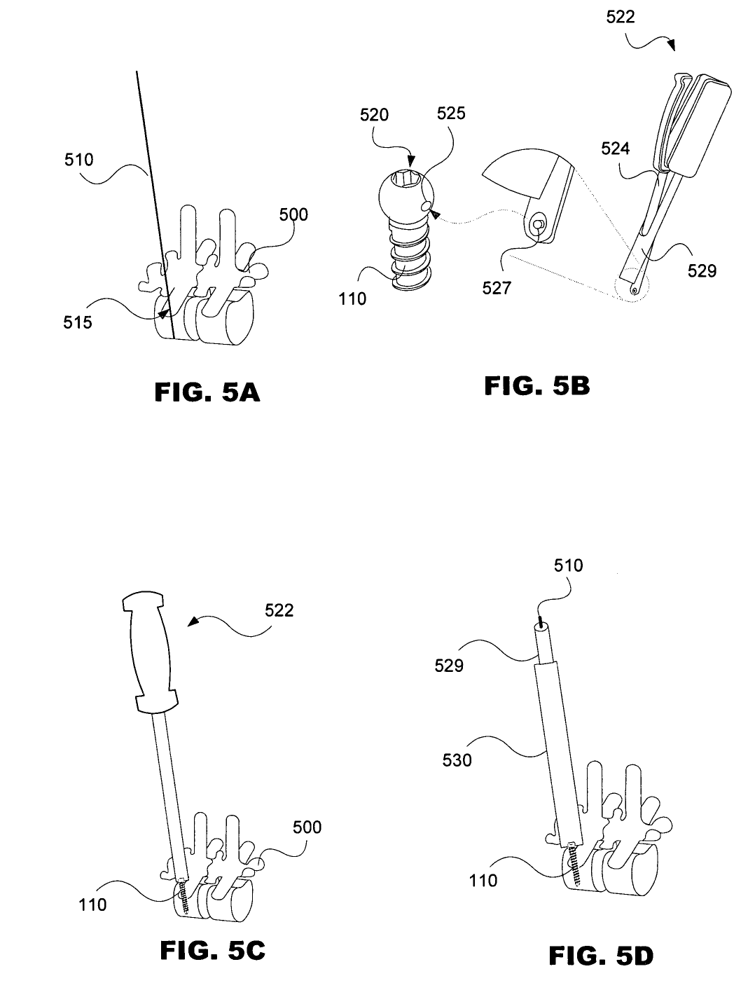

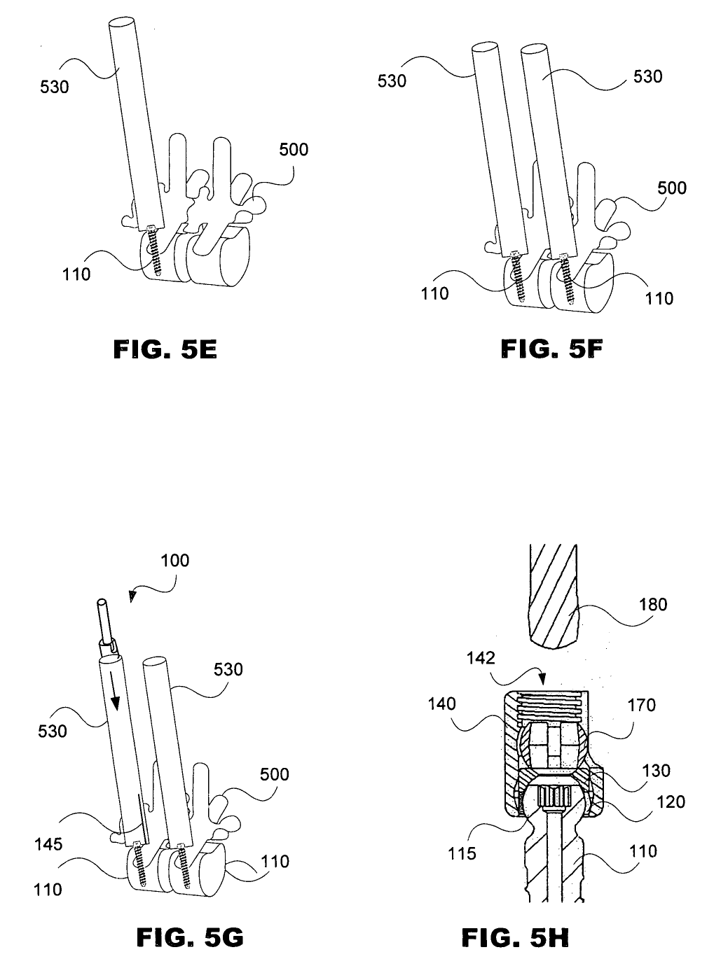

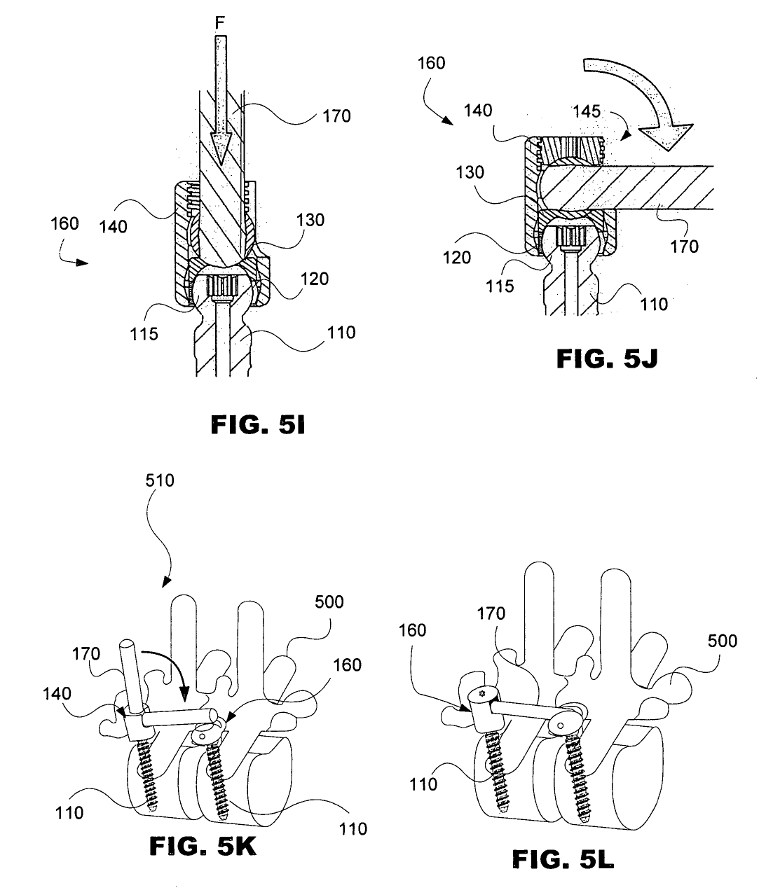

FIG. 4 illustrates an exemplary percutaneous rod placement method that may be performed with the percutaneous pedicle screw system (100) of FIG. 1, according to one exemplary embodiment. As illustrated in FIG. 4, the exemplary method begins by first incising a patient and placing a K-wire into a desired pedicle (step 400). Then, a pedicle screw is placed in the desire pedicle using the K-wire as a guide (step 405). With the pedicle screw in place, a percutaneous tube may be placed over the pedicle screw to the level of the desired pedicle (step 410). Steps 400 through 410 may then be repeated on a second desired pedicle (step 415) until all the desired pedicles have pedicle screws securely placed and percutaneous tubes providing access thereto. A percutaneous pedicle screw tulip and connector rod may then be passed down the percutaneous tube and the tulip may be snapped onto a first pedicle screw head (step 420). The connector rod may then be rocked over onto the head of an adjacent tulip through slots in the percutaneous tubes along the fascial plane lateral to the multifidus (step 425). When the rod is secured in an adjacent tulip, the percutaneous tubes may be removed (step 425) and the wounds treated. The above-mentioned method will be described in detail below with reference to FIGS. 4 through 5L.

As mentioned above, the exemplary method begins by first incising a patient and placing a K-wire into a desired pedicle (step 400). FIG. 5A illustrates placement of a K-wire (510) into a pedicle (515) of an identified vertebra (500). According to one exemplary embodiment, placement of the K-wire may be achieved by performing a blunt dissection in the plane lateral to the multifidus approaching the pedicle (515). The lumbar vertebrae (500) have a number of muscle groups that run on top of the vertebra. The multifidus muscle is located adjacent to the spinous process with the longissimus muscle group being positioned lateral to the multifidus. In contrast to the present exemplary method, traditional MIS approaches insert K-wires, pedicle screws, and their associated hardware through an entry path that traverses the multifidus muscle group. This technique unnecessarily damages soft tissue, resulting in pain and increased rehabilitation for the patient. The blunt dissection and insertion of the K-wire may be facilitated by fluoroscopic guidance. Further details of the insertion technique by performing a blunt dissection in the plane lateral to the multifidus approaching the pedicle (515) may be found in U.S. patent application entitled “Less Invasive Access Port” filed Mar. 17, 2006 by David T. Hawkes et al., attorney docket number 40359-0070, the application is incorporated herein by reference in its entirety.

With the K-wire in place, a pedicle screw is placed in the desire pedicle using the K-wire as a guide (step 405; FIG. 4). FIGS. 5B and 5C illustrate an exemplary tool and method of inserting the pedicle screw in the desired pedicle using the K-wire as a guide (step 405; FIG. 4). According to one exemplary embodiment, the K-wire may be used as a guide to drill and tap the desired pedicle (515). Once prepared, the pedicle screw (110; FIG. 1) may be driven into the desired pedicle (515) with a screw driver (5220).

As illustrated in FIG. 5B, an exemplary screw driver (522) including a stationary driving arm (529) and a pivotable driving arm (524) may be used to place the pedicle screw. According to one exemplary embodiment, the screw head (115) may contain a traditional driving feature (520) and a drive reception orifice (525) through the sides which mates with a drive protrusion (527) in the tip of the pivotable driving arm (524). With the exemplary screw driver (522) illustrated in FIG. 5B, the tulip-rod assembly may first assembled with the pedicle screw and the set screw (150; FIG. 1) partially tightened to capture the pedicle screw (110; FIG. 1) within the tulip (160; FIG. 1) without rigidly locking it. A cannulated rod may then be slipped into the shaft of the driver and the handle closed, engaging the pin into the head of the pedicle screw to securing it. The screw assembly can then be driven and released through a 15.5 mm percutaneous tube.

Returning again to the exemplary method of FIG. 4, once the pedicle screw is in place, a percutaneous tube may be placed over the pedicle screw to the level of the desired pedicle (step 410). As shown in FIG. 5D, the handle of the driver (522) may be removed, allowing the percutaneous tube (530) to be placed directly over the driving arm (529) down to the level of the pedicle (515). As illustrated in FIG. 5E, when the percutaneous tube (530) is properly placed, the driving arm (529) and the K-wire (510) are removed, leaving the pedicle screw (110) and the percutaneous tube (530) in place. With the first location prepared, steps 400 through 410 may then be repeated on a second desired pedicle (step 315) until all the desired pedicles have pedicle screws securely placed and percutaneous tubes providing access thereto. FIG. 5F illustrates the performance of steps 400 through 410 on a second desired pedicle, according to one exemplary embodiment.

With one or more percutaneous tubes in place (530), a percutaneous pedicle screw tulip and connector rod may then be passed down the percutaneous tube and the tulip may be snapped onto a first pedicle screw head (step 420). According to the exemplary embodiment illustrated in FIG. 5G, the percutaneous screw tulip is assembled to a connector rod to form an assembled percutaneous pedicle screw system (100) and passed down the percutaneous tube (530) tulip first. However, as illustrated in FIGS. 5H and 5I, the tulip assembly (160) may first be coupled to the head (115) of the pedicle screw (110), followed by a coupling of the rod (170) to the tulip assembly (160). As shown in FIGS. 5H and 5I, the rod (180) may be guided down the percutaneous tube (530) where it engages the inner bore (142) of the tulip housing (140). According to one exemplary embodiment the tulip assembly (160) is supplied with the split ball end (170) pre-assembled. Once introduced into the inner bore (142), a force (F) introduces the rod (180) into the split ball end (170) to retain the rod.

With the connector rod (180) inserted into the tulip assembly (160) and coupled to the pedicle screw (110), the connector rod may then be rocked over onto the head of an adjacent tulip through slots in the percutaneous tubes along the fascial plane lateral to the multifidus (step 325). FIGS. 5J through 5F illustrate the connector rod (180) being rocked over on to the head of an adjacent tulip assembly (160). As mentioned above, the tulip housing (140) includes a rod cut out (145) in the side wall thereof. Additionally, the percutaneous tubes may include a slit in the wall thereof (510) to allow for rotation of the rod (180). According to one exemplary embodiment, the rod (180) is rocked over, passed under the patient's skin along the fascial plane lateral to the multifidus, until it engages an adjacent tulip assembly (160). Once the second tulip assembly (160) is engaged, both tulip assemblies may be locked into place by securing the set screw (150) in the inner bore (142) to seat the split ball end (170) in the saddle (130). Alternatively, the adjacent tulip (160) may include any number of other locking mechanisms for securely locking the connector rod in place.

When the rod is secured in an adjacent tulip, the percutaneous tubes may be removed (step 425) and the wounds treated. FIG. 5L illustrates a fully assembled construct with the percutaneous tubes (530) removed. According to the present exemplary embodiment, the only surface wounds that will be treated are the wounds formed to allow the insertion of the percutaneous tubes. The placement of the rod is performed under the skin, eliminating a great deal of paraspinous tissue damage.

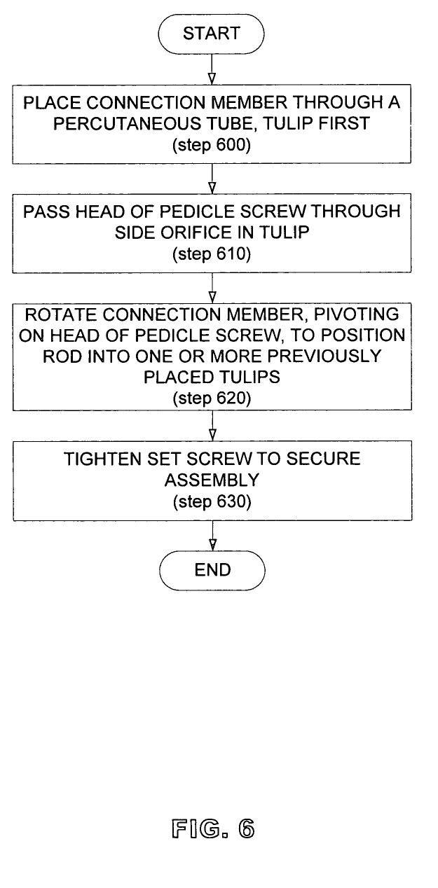

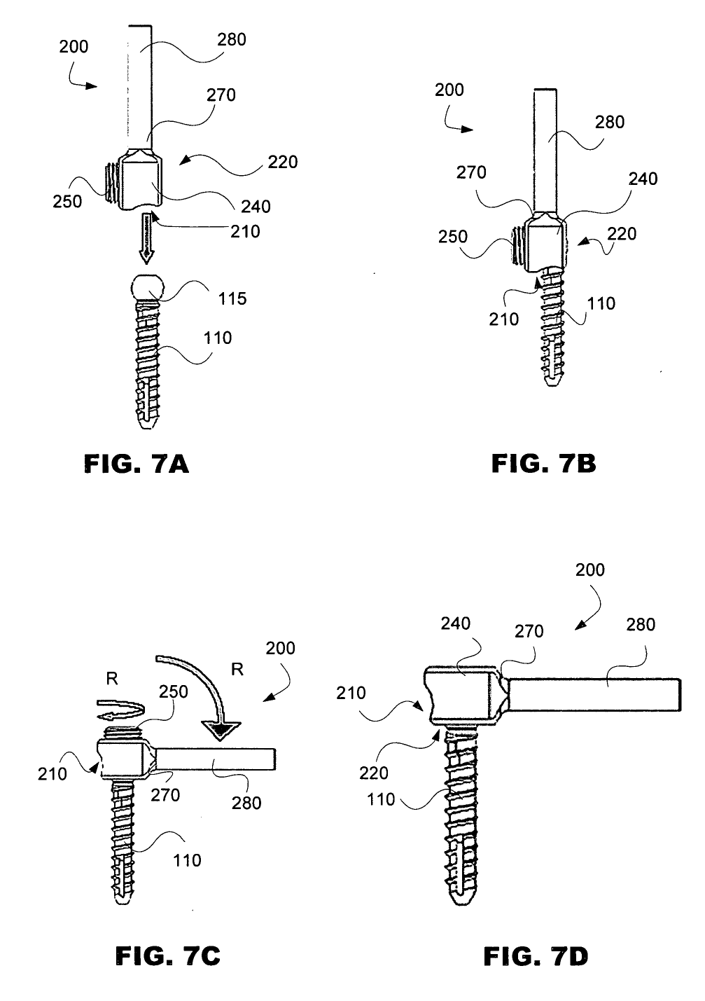

The method illustrated in FIG. 4 may also be used to insert the alternative percutaneous pedicle screw system (200) of FIG. 2. As illustrated in FIGS. 6 through 7D. As shown, once the percutaneous tubes (530; FIG. 5E) are in place, the connection member may be place through the percutaneous tube, tulip first (step 600), as illustrated in FIG. 7A. Once presented to the head portion (115) of the pedicle screw (110), the head of the pedicle screw may be passed through the side orifice (210) in the tulip (step 610), as shown in FIG. 7B.

In contrast to the first percutaneous pedicle screw system (100; FIG. 1) which only rotates the rod (180), the second exemplary percutaneous pedicle screw system (200) rotates the entire percutaneous pedicle screw system, pivoting on the head of the pedicle screw, to position the rod into one or more previously placed tulips (step 620). As shown in FIG. 7C, rotation of the system causes the threaded portion of the pedicle screw (110) to be exiting the bottom orifice (220) of the tulip housing (240). Similar to the first exemplary percutaneous pedicle screw system (100; FIG. 1), the rod portion (280) is passed through a slit (510; FIG. 5K) in the wall of the percutaneous tube (530; FIG. 5K) to allow for rotation of the rod (280). According to one exemplary embodiment, the rod (280) is rocked over, passed under the patient's skin along the fascial plane lateral to the multifidus, until it engages an adjacent tulip assembly.

Once the second tulip assembly is engaged, the set screw (250) is tightened to secure the assembly (step 630). As mentioned previously and as shown in FIGS. 7C and 7D, tightening of the set screw (250) seats the head portion (115) of the pedicle screw (110) in the seating taper (225; FIG. 2) of the thru-bore (310). Additionally, tightening of the set screw (250) obstructs the head reception orifice (210), securely retaining the head of the pedicle screw.

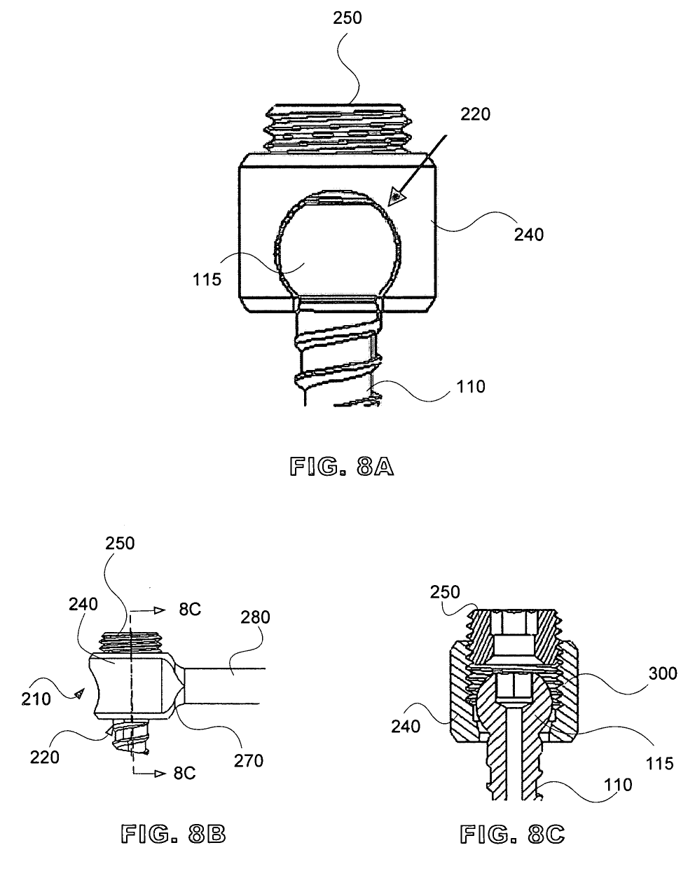

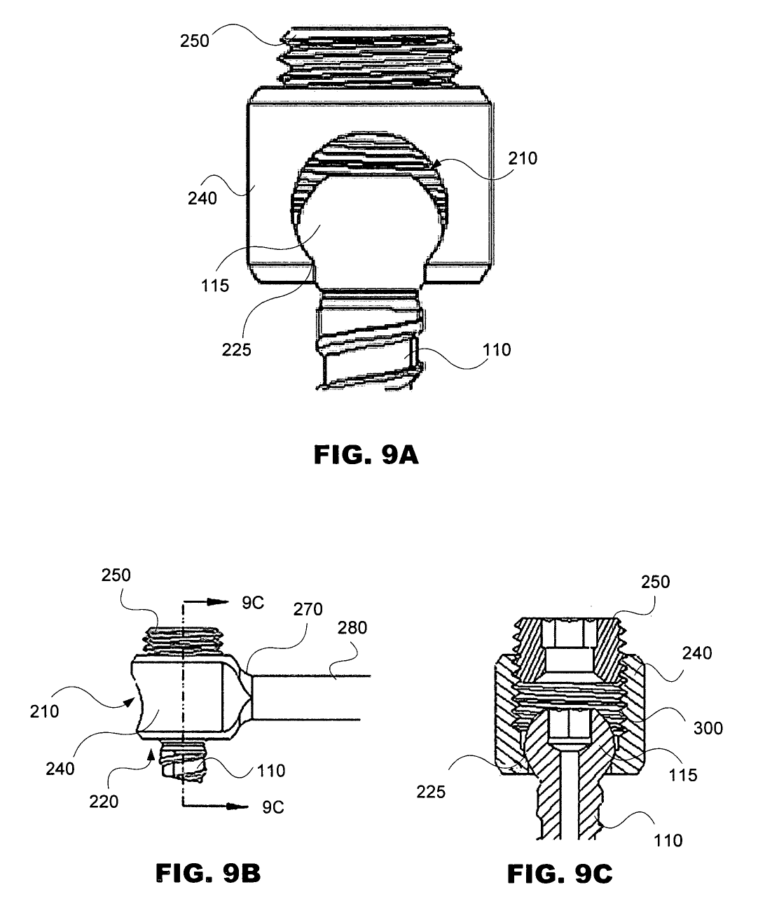

FIGS. 8A through 10C illustrate the seating of the head portion (115) of the pedicle screw (110), according to one exemplary embodiment. As shown in FIGS. 8A-8C, prior to engagement of the set screw (250), the spherical screw head (115) is passed through the head reception orifice (210) in the back of the tulip into the center of the tulip and positioned such that the thread portion of the pedicle screw (110) is exiting the bottom orifice (220) of the tulip housing (240). When correctly positioned, the screw head (115) is then seated in the spherical seating taper (225) in line with the axis of the set screw (250), as illustrated in FIGS. 9A-9C.

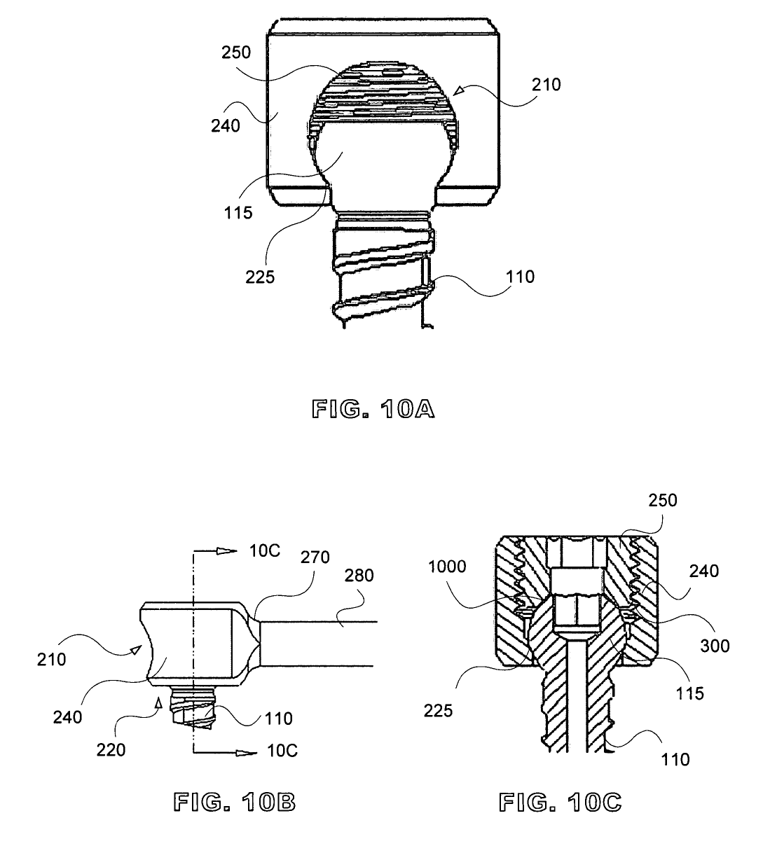

The set screw (250) is then advanced down the thru-bore (310; FIG. 3B) to engage the screw head (115), locking it into the seating taper (225). As illustrated in FIGS. 10A-10C, the set screw (250) may have a concave head receiving surface (1000) configured to mate with the upper surface of the screw head (115), thereby constraining the construct in the lateral plane. Additionally, the advancement of the set screw (250) against the head portion (115) of the pedicle screw (110) positionally locks the exemplary percutaneous pedicle screw system (200) relative to the pedicle screw.

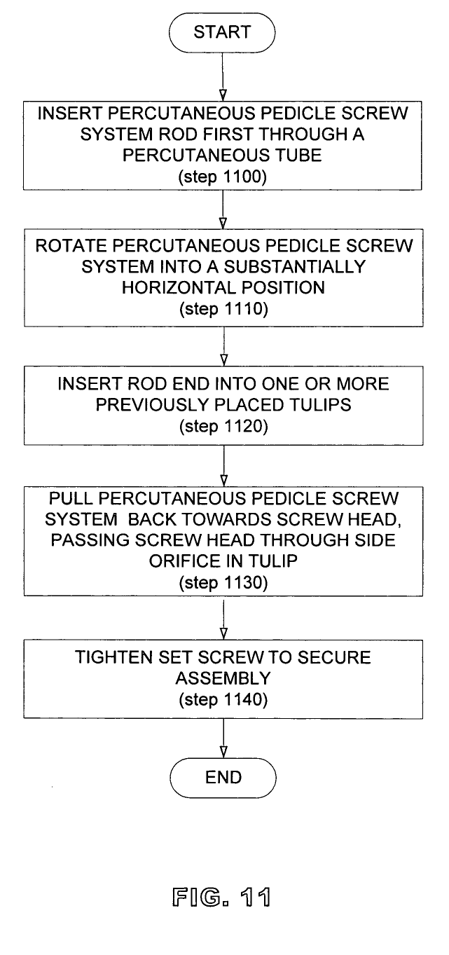

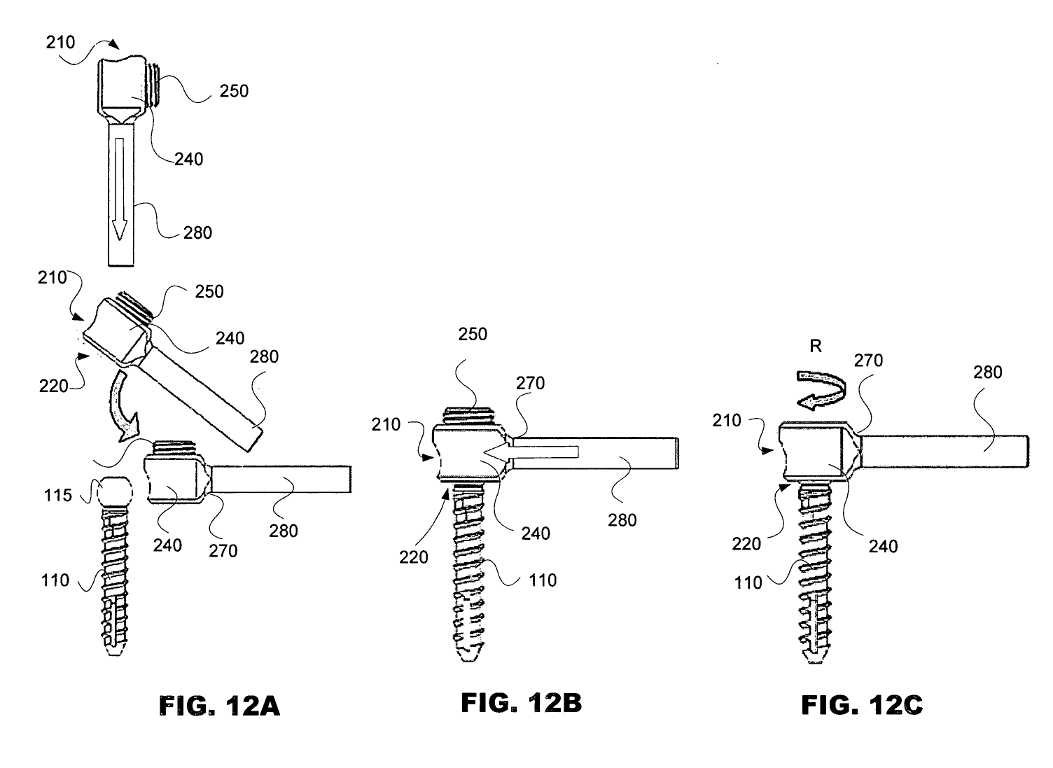

The above-mentioned insertion methods allow for the insertion and fixation of the screw assemblies subcutaneously, due to the short rod requirement of a one level coupling. Particularly, when coupling only two vertebra, the rod used is sufficiently short to allow for the assembly to be inserted tulip first, followed by the rod being rocked over, subcutaneously. However, 2 or 3 level procedures that couple more than 2 vertebra incorporate rods having greater lengths. Consequently, FIGS. 11 through 12C illustrate an exemplary rod-first insertion method that may be used for 2 or 3 level procedures.

As illustrated in FIG. 11, the exemplary method begins, after insertion of the percutaneous tubes (530; FIG. 5D) and the pedicle screws (110), as described above, by inserting the percutaneous pedicle screw system rod first through the percutaneous tubes (step 1100) followed by rotating the percutaneous pedicle screw system into a substantially horizontal position (step 1110). FIG. 12A illustrates such an insertion. As shown, the percutaneous pedicle screw system (200) is inserted with the rod (280) at the leading edge. As the system (200) is passed near the head portion (115) of the pedicle screw (110), the percutaneous pedicle screw system is rotated, along the fascial plane lateral to the multifidous, into a substantially horizontal position. As shown in FIG. 12A, the head reception orifice (210) of the tulip housing (240) will then be substantially adjacent to the head portion (115) of the pedicle screw (110).

Either as the system is being placed into a substantially horizontal position, or thereafter, the rod (280) can be inserted into one or more previously placed tulip assemblies (step 1120). The tulip assembly may then be coupled to the head portion (115) of the pedicle screw (110) by pulling the percutaneous pedicle screw system (200) back towards the head portion of the screw, passing the screw head through the side orifice in the tulip (step 1130). FIG. 12B illustrates the insertion of the head portion into the tulip assembly. If the first percutaneous pedicle screw system (100; FIG. 1) is being used, the tulip assembly may be lifted above the head portion (115) of the pedicle screw (110) as the system is pulled back. With the percutaneous pedicle screw system properly positioned, the set screw may then be tightened to secure the assembly (step 1140), as illustrated in FIG. 12C.

In conclusion, the present exemplary percutaneous pedicle screw systems and methods provide a number of exemplary connection members and methods that can be used for pecutaneous screw placement. Specifically, the present exemplary systems and methods provide for the percutaneous placement of pedicle screws, followed by easy placement of the rod and one or more tulips simultaneously via a percutaneous tube. Specifically, the present exemplary system and method allows a surgeon to place spinal screws and rods via a true percutaneous approach by providing for pivoting of the rod beneath the skin in a fascial plane, lateral to the multifidous. Using the disclosed MIS approach to spinal fixation and/or correction surgery will effectively decrease a patient's recovery time and reduce the risks of follow-up surgeries.

It will be understood that various modifications may be made without departing from the spirit and scope of the present exemplary systems and methods. For example, while the exemplary implementations have been described and shown using screws to anchor into bony structures, the scope of the present exemplary system and methods is not so limited. Any means of anchoring can be used, such as a cam, screw, staple, nail, pin, or hook.

The preceding description has been presented only to illustrate and describe embodiments of invention. It is not intended to be exhaustive or to limit the invention to any precise form disclosed. Many modifications and variations are possible in light of the above teaching. It is intended that the scope of the invention be defined by the following claims.

What is claimed is:

1. A connection member for percutaneously coupling to one or more orthopedic fasteners comprising:
a tulip assembly; and
a rod, wherein said rod is permanently coupled to said tulip assembly.

2. The connection member of claim 1, wherein said tulip assembly and said rod are a single continuous member.

3. The connection member of claim 1, wherein said rod is permanently coupled to said tulip assembly by an attaching element.

4. The connection member of claim 3, wherein said attaching element comprises one of a threaded interface, a weld, or an adhesive.

5. A connection member for percutaneously coupling to one or more orthopedic fasteners comprising:
a fastener head securing member including a fastener head securing orifice having an axis defined by a wall member, said wall member defining a head retention orifice on an end of said head securing orifice;
an adjustable compression member coupled to a surface of said wall member;
a rod coupled to said wall member; and
a fastener head receiving orifice formed in said wall member, wherein said fastener head receiving orifice is formed transverse to and intersects an axis of said fastener head securing orifice.

6. The connection member of claim 5, further comprising a seating taper formed on said wall member proximal to said head retention orifice.

7. The connection member of claim 5, wherein said rod is coupled to said wall member by one of a weld, an adhesive, or a threaded system.

8. The connection member of claim 5, wherein said adjustable compression member comprises a set screw.

9. The connection member of claim 5, wherein:
said head retention orifice has an outer diameter less than an outer diameter of said fastener head; and
said fastener receiving orifice has a diameter greater than an outer diameter of said fastener head.

10. A bone fixation device comprising:
a screw, said screw including a threaded portion, a spherical head, and a driving interface;
a tulip assembly configured to be coupled to said spherical head of said screw, wherein said tulip assembly includes an outer housing defining a thru-bore, a split ball, a saddle, and a split ring disposed in said thru-bore, a plurality of grooves formed on an upper surface of said thru-bore, and a cutout extending from a top of said thru-bore down to a selected distance along a side of said outer housing; and
a set screw having an outer surface and a ridged outer perimeter surface, wherein said ridges on said ridged outer perimeter mateably connect to said plurality of grooves formed on an upper surface of said thru-bore.

11. The bone fixation device of claim 10, wherein said plurality of grooves formed on an upper surface of said thru-bore and said ridges on said ridged outer perimeter of said set screw comprise threads.

12. The bone fixation device of claim 10, further comprising a rod configured to be coupled to said tulip assembly, wherein said cutout is configured to receive a largest outer diameter of said rod.

13. The bone fixation device of claim 10, wherein said split ball is configured to be coupled to an end portion of said rod.

14. The bone fixation device of claim 12, further comprising corresponding features on an inner surface of said split ball and an outer surface of said rod, said corresponding features being configured to couple said split ball to said outer surface.

15. The bone fixation device of claim 14, wherein said corresponding features comprise one of apposing tapers, single or multiple radial grooves, or threading.

16. The bone fixation device of claim 10, further comprising:
a split ring receiving bore defined in said thru-bore;
wherein said split receiving bore has an outer diameter associated with an outer diameter of said split ring when said split ring is expanded around said spherical head;
said tulip assembly being configured to snap onto said spherical head of said screw.

17. The bone fixation device of claim 10, wherein:
said saddle is oriented adjacent to said split ring inside said thru-bore;
said split ball is disposed on said saddle;
wherein a downward force exerted on said split ball is transferred to said saddle.

18. A method for coupling a connection member including a tulip to at least one orthopedic fastener having a fastening shaft comprising:
passing a head of said orthopedic fastener through a first orifice in said connection member along a first line of motion;
orienting said connection member with respect to said orthopedic fastener such that said fastening shaft is oriented perpendicular to said first line of motion;
seating said orthopedic fastener head in said connection member; and
positionally fixing said orthopedic fastener in said connection member.

19. The method of claim 18, wherein said coupling of said connection member to said at least one orthopedic fasteners is performed percutaneously.

20. The method of claim 19, wherein said passing a head of said orthopedic fastener through a first orifice comprises:
securing said orthopedic fastener in a bone member; and
passing said connection member through a percutaneous tube, tulip first.

21. The method of claim 19, wherein said passing a head of said orthopedic fastener through a first orifice comprises:
securing said orthopedic fastener in a bone member; and
passing said connection member through a percutaneous tube, rod first.

22. A method for installing a percutaneous tulip assembly comprising:
installing a K-wire into a desired pedicle, wherein said K-wire is installed along a fascial plane proximal to a multifidous muscle;
driving a pedicle screw into said desired pedicle using said K-wire as a guide;
inserting a percutaneous tube along said K-wire; and
inserting said percutaneous tulip assembly onto said pedicle screw via said percutaneous tube.

23. The method of claim 22, further comprising:
coupling a rod to said percutaneous tulip prior to inserting said tulip; and
positioning said rod and tulip combination in said percutaneous tube such that said rod protrudes upward in said tube.

24. The method of claim 23, further comprising rotating said rod and tulip combination to engage said rod with a second tulip assembly.

25. The method of claim 24, further comprising rotating said rod through a slit in said percutaneous tube.

26. The method of claim 25, further comprising passing said rod through said fascial plane proximal to said multifidous muscle when said rod is rotated to engage said second tulip assembly.

27. A percutaneous tube comprising:
a tube including a proximal end and a distal end, said tube defining an inner passage; and
a separation formed in a wall of said tube on said distal end, said separation being configured to permit passage of a rod.

28. A screw-driving device comprising:
a handle;
a first driving member fixedly coupled to said handle;
a second driving member pivotably coupled to said first driving member; and
a screw engagement feature disposed on one end portion of said second driving arm.

29. The screw-driving device of claim 28, wherein said first driving member comprises a hollow shaft defining an inner space, wherein said inner space is configured to house a tulip and a rod when said screw-driving device is driveably coupled to a screw.

30. The screw-driving device of claim 29, wherein said screw comprises a thread portion and a head portion, said head portion comprising a cylindrical orifice;
wherein said screw engagement feature includes a cylindrical protrusion configured to mateably engage said cylindrical orifice.
